(19) United States
(12) Patent Application Publication
(10) Pub. No.: US 2006/0154891 A1
(43) Pub. Date: Jul. 13, 2006

(54) Ready-to-use gemcitabine solutions and gemcitabin concentrates

(76) Inventors: Edgar Schridde, Hannover (DE); Stefan-Peter Gimmel, Hannover (DE); Bernd Merbach, Burgwedel (DE)

Correspondence Address: SCHWEITZER CORNMAN GROSS & BONDELL LLP, 292 MADISON AVENUE - 19th FLOOR, NEW YORK, NY, 10017, US

(21) Appl. No.: 11/316,174

(22) Filed: Dec. 22, 2005

(30) Foreign Application Priority Data
Dec. 23, 2004 (DE) 10 2004 063 347.9

(51) Int. Cl. A61K 31/7072; B65D 85/42

(52) U.S. Cl. 514/49; 206/528

(57) ABSTRACT

Pharmaceutical compositions in the form of ready-to-use preparations of gemcitabine in aqueous solutions in a glass containers having specified dimensional relationships demonstrate long shelf life over a wide range of solution pH values. The ratio of the surface area of the container wetted by the solution to the volume of the solution, expressed in cm² to cm³, is less than 3.4.

The present invention relates to pharmaceutical compositions, containing gemcitabine, in the form of ready-to-use solutions.

### BACKGROUND OF THE INVENTION

Gemcitabine (2′-deoxy-2′,2′-difluorocytidine; 1-(4-amino-2-oxo-1H-pyrimidine-1-yl)-2-deoxy-2,2-difluororibose; dFdC; CAS No. 95058-81-4; C₉H₁₁F₃N₃)₄, MW 263.2) is a substance, which is officially monographed in the Pharmacopoeia (Official Monographs, USP 27, first supplement USP—NF, page 3060-61 with respect to “Gemcitabine Hydrochloride” and “Gemcitabine for Injection”) and has the chemical structure

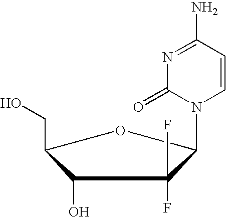

Gemcitabine was described for the first time in U.S. Pat. No. 4,526,988 and can be used for the treatment of viral infections or in immunosuppressive therapy of autoimmune diseases. The anti-neoplastic effectiveness of gemcitabine is disclosed in U.S. Pat. No. 5,464,826. It is used therapeutically alone, and also in combination with other cytostatic agents such as cisplatin for the treatment of locally advanced or metastasized, not small-celled bronchial cancer as well as advanced adenocarcinoma or cysadenocarcinoma of the exocrine pancreas. The recommended dosage in gemcitabine therapy usually is 1 g per square meter of body surface. Like other nucleoside analogs, gemcitabine can also be used cytostatically for the therapeutic treatment of different types of cancer, such as lymphatic or myeloic leukemia. For the treatment of different types of cancer, gemcitabine is administered intravenously, so that it is necessary to make the active ingredient available in the form of a solution.

At the present time, gemcitabine preparations required for parenteral administration are available only in the form of lyophilisates (Gemzar®), which must be reconstituted before being administered to the patient. However, the use of such freeze-dried preparations harbors significant disadvantages. On the one hand, the process of manufacturing the lyophilisate is complicated and expensive and, on the other, the reconstitution represents additional steps and entails undesirable risks for the personnel concerned therewith. In particular, during the reconstitution of the drug solution from a dry substance, there may be a so-called “spray-back effect”, which may result in further contamination and danger to the personnel. Accordingly, during the preparation of the lyophilisate as well as during its reconstitution, any contamination of personnel or inventory with the highly effective cytostatic agent must be avoided. Moreover, serious problems during treatment with gemcitabine, such as a deviation in the concentration of the active ingredient or microbial contamination of the solution produced from the lyophilisate, may also arise due to errors in handling the lyophilisate.

Because of the various sources of danger and errors during the use of a lyophilized active ingredient for the preparation of gemcitabine solutions, it is therefore desirable to have ready-to-use solutions of the drug available.

As is well known, gemcitabine solutions presently used and reconstituted from lyophilisates are not stable when stored, since they are subject to degradation of the active ingredient during storage. On the one hand, this leads to a deviation in the concentration of active ingredient and, on the other, to undesirable contamination (decomposition product of the gemcitabine) in the solution.

It is an object of the present invention to make available stable, ready-to-use gemcitabine solutions, which do not have the above-discussed risks and disadvantages of known forms of administration and of solutions produced therefrom. In this connection, the expression, “ready-to-use”, means that the solution is not reconstituted from a solid, such as a crystalline or an amorphous solid or a lyophilisate, immediately before it is administered to a patient.

At room temperature, gemcitabine hydrochloride forms a clear solution at a concentration of about 40 mg/milliliter in a strong, aqueous hydrochloric acid solution (pH approximately 3, reconstituted Gemzar® lyophilisate, Eli Lilly) or at a concentration of about 15 mg/mL in an aqueous, weakly alkaline solution having a pH of about 8, and at a concentration of up to 110 mg/mL in an alkaline, ethanolic, aqueous solution with a pH of approximately 10.

In published European patent application EP 1 479 389 A1, the stability of gemcitabine in an aqueous solution was investigated in the pH range from 3 to 10, a pH of about 8 being described as optimum.

Since gemcitabine solutions in oncological therapy are usually infused in a dose of 1000 mg/m² of body surface within a period of 30 minutes, it is necessary to make available the volume of solution, required for this purpose, in a pharmaceutically suitable container.

For example, for a person to be treated whose height is 180 cm and whose weight is 80 kg and who therefore has a body surface area of 2 m², a total of 2000 mg of gemcitabine is administered.

Different solution concentrations and solution volumes are available, such as a 200 mL gemcitabine solution containing 10 mg/mL or a 40 mL ethanolic aqueous gemcitabine concentrate containing 50 mg/mL or a 25 mL ethanolic aqueous gemcitabine solution containing 80 mg/mL, as volumes to be prepared in suitable packaging means.

Gemcitabine as an active ingredient (Gemzar® lyophilisate) is also indicated at the present time for the treatment of pancreatic cancer as well as, in combination with the active ingredient paclitaxel, for the treatment of breast cancer and, in combination with the active ingredient cisplatin, for the treatment of not small-celled lung cancer.

Usually, these combinations of active ingredients, as well as other therapeutic solutions of accompanying active ingredients, are administered consecutively and intravenously through the same injection port (a so-called Y-site injection), attention having to be paid to the compatibility of the solutions administered in order to avoid precipitation of active ingredients, since such precipitates, as infused particles, may be life threatening. Accordingly, information concerning the compatibility of gemcitabine solutions has been published, for example, in the “Handbook on Injectable Drugs”, 12th Ed., Lawrence A. Trissel, pages 653-661, in the context of its compatibility with different carrier solutions for infusion as well as its compatibility with different solutions of active ingredients (Y-Site Injection Compatibility, 1:1 mixture). Information concerning the use of Gemzar® itself does not provide any information concerning the compatibility of Gemzar® with other active ingredients.

However, on the part of the user there is also much interest in administering gemcitabine in combination with other cytostatically active ingredients in a so-called infusion cocktail of cytostatic agents (a mixed infusion solution), in order to take advantage of the therapeutically intended synergism of action and, with that, to increase the effectiveness of the treatment, as well as to avoid burdening the patient with time-consuming individual infusions of previously prepared or ready-to-use solutions of individual active ingredients, so as to improve the quality of life of the patient. Moreover, the combined administration as a mixed infusion can clearly increase the possibilities of the treating physician in clinical practice within the scope of his treatment freedom, especially in connection with the option of clinical tests with new active ingredients, which are still unknown at the present time.

However, these active ingredients always have individual chemical and physical properties which must be taken into consideration and may differ from those of gemcitabine, for example, with regard to their solubility, their pH for optimum stability and their physical and chemical compatibility with other active ingredients. These individual properties, in turn, are determined especially by the pH in an aqueous solution.

In conjunction with the preparation of infusion cocktails of cytostatic agents by mixing solutions of individual active ingredients with their different properties, especially with deviating pH values, physically and/or chemically unfavorable pH values may result, which can bring about, for example, an undesirably reduced stability, an undesirable incompatibility of the active ingredients with one another, or a crystallization of particles from the mixture of solutions, which may be life-threatening to a patient if such solutions are administered.

In this connection, it may therefore be of advantage to make available ready-to-use gemcitabine solutions and gemcitabine concentrates with a pH that differs from a pH of 8, which is particularly advantageous for gemcitabine. Such solutions may, for example, be acidic, ready-to-use gemcitabine solutions and concentrates, for example, with a pH of 5 or basic, ready-to-use gemcitabine solutions and concentrates, for example, with a pH of 10, so that the above-mentioned undesirable disadvantages in connection with the preparation and administration of gemcitabine solutions alone or combined as so-called mixed infusion solutions can be avoided.

Unfortunately, gemcitabine solutions with a pH deviating from the particularly advantageous value of 8 (such as a pH of 5 or 10) have clearly low stabilities and consequently shorter shelf lives at the preferred storage temperature of 25° C. This is disadvantageous for marketing.

It is therefore an object of the present invention to make available gemcitabine-containing ready-to-use solutions and concentrates that avoid these risks and disadvantages and are stable over a larger range of pH values, so that they permit the long-term use of ready-to-use gemcitabine solutions and concentrates as therapeutic agents.

A further object of the present invention is to make available gemcitabine-containing ready-to-use solutions and concentrates that avoid the above-mentioned disadvantages in that suitable, stable, gemcitabine solutions and concentrates with more acidic and basic pH values than a pH of about 8 are made available in containers suitable for this purpose.

Yet another object of the present invention is to provide glass containers having the appropriate characteristic values that are an essential prerequisite for achieving a shelf life of 36 months at a temperature 25° C., during which the content of the active ingredient does not fall below 95% of the initial content.

### BRIEF DESCRIPTION OF THE INVENTION

In experiments upon which the present invention is based it was surprisingly found that ready-to-use solutions containing gemcitabine with a pH deviating from the particularly advantageous value of 8, for example with a pH of 5 or 10, show shelf lives at 25° C., which are more suitable for marketing, if they are filled into and stored in tightly closed glass containers, which have dimensions recited herein.

The glass containers suitable for this purpose are distinguished by geometric proportions, with preferred containers having a ratio of vessel surface (A) wetted with gemcitabine active ingredient solution to the volume (V) of the gemcitabine solution filled into the container of less than about 3.4 (cm²/cm³). Particularly preferred are those vessels with an A/V ratio of less than about 2.8 and especially of less than about 2.4 (cm²/cm³).

Likewise preferred are glass vessels for which the ratio of the total surface area of the vessel (AT) to the full-to-the-edge or top capacity (RV) is less than 4.6 (cm²/cm³). Particularly preferred are those vessels with an AT/RV ratio of less than about 4.0 (cm²/cm³) and, in particular, of less than about 3.3 (cm²/cm³).

The inventive vessels are made from glass of a type that is suitable for injectables. Particularly preferred packing means, in general, are glass containers such as the EN ISO 8362 glass vials from glass tubing of type I, EN ISO 8362 glass vials from blanks of type I1 or EN ISO 58363-5 infusion flasks from blanks of type I.

### BRIEF DESCRIPTION OF THE DRAWINGS

A fuller understanding of the invention will be achieved upon consideration of the following detailed discussion and description of preferred and illustrative examples thereof, when reviewed in association with the annexed drawings, wherein:

FIG. 1 is a graph of the stability of a 10 mg/mL gemcitabine solution at pH 5 as a function of the surface area/volume ratio for a glass container storing the solution in accordance with the invention;

FIG. 2 is a graph of the stability of a 10 mg/mL gemcitabine solution at pH 8 in such a container as a function of the container surface area/volume ratio;

FIG. 3 is a graph of the stability of a 10 mg/mL gemcitabine solution at pH 10 in such a container as a function of the container surface area/volume ratio;

FIG. 4 is a graph of the stability of a 50 mg/mL gemcitabine solution at pH 8 in such a container as a function of the container surface area/volume ratio;

FIG. 5 is a graph of the stability of a 50 mg/mL gemcitabine solution at pH 10 in such a container as a function of the container surface area/volume ratio;

FIG. 6 is a graph of the stability of a 10 mg/mL gemcitabine solution at pH 5 as a function of the surface area/full-to-the-edge capacity ration of a glass container storing the solution in accordance with the invention;

FIG. 7 is a graph of the stability of a 10 mg/mL gemcitabine solution at pH 8 in such a container as a function of the container surface area/full-to-the-edge capacity ratio;

FIG. 8 is a graph of the stability of a 10 mg/mL gemcitabine solution at pH 10 in such a container as a function of the container surface area/full-to-the-edge capacity ratio;

FIG. 9 is a graph of the stability of a 50 mg/mL gemcitabine solution at pH 8 in such a container as a function of the container surface area/full-to-the-edge capacity ratio; and

FIG. 10 is a graph of the stability of a 50 mg/mL gemcitabine solution at pH 10 in such a container as a function of the container surface area/full-to-the-edge capacity ratio.

### DETAILED DESCRIPTION OF THE INVENTION

In the following Table 1, the calculated ratio of surface area to volume (A/V) is shown by way of example for containers of various nominal capacities V, the nominal capacity or volume V being defined as the total volume of the gemcitabine solution and A as the calculated surface area of the glass container wetted by the gemcitabine solution.

TABLE 1
Parameters of selected glass containers

| Nominal | | Filling Height | | |
|---|---|---|---|---|
| Capacity | d | of Solution | A | A / V |
| V | (cm) | h (cm) | (cm²) | (cm²/cm³) |
| 2 ml¹⁾ | 1.60 | 1.00 | 7.04 | 3.52 |
| 4 ml¹⁾ | 1.60 | 1.99 | 12.01 | 3.00 |
| 10 ml¹⁾ | 2.40 | 2.21 | 21.19 | 2.12 |
| 20 ml¹⁾ | 3.00 | 2.83 | 33.74 | 1.97 |
| 30 ml²⁾ | 3.60 | 2.95 | 43.54 | 1.45 |
| 50 ml³⁾ | 4.25 | 3.52 | 61.19 | 1.22 |
| 100 ml²⁾ | 5.16 | 4.78 | 98.40 | 0.98 |
| 250 ml³⁾ | 6.60 | 7.31 | 185.73 | 0.74 |

¹⁾EN ISO 8362-1; Glass vial of tubular glass Type I
²⁾EN ISO 8362-1; Glass vial from glass blanks Type I
³⁾EN ISO 58363-5 Infusion bottles from glass blanks Type I

In Table 2, the calculated ratio of the surface area to the capacity (AT/RV) is shown by way of example for containers of nominal capacity RV, the capacity RV being defined as the full-to-the-edge capacity of the glass container and AT as the calculated surface area of the glass container.

TABLE 2
Parameters of selected glass containers

| Capacity filled | | Total height of | | |
|---|---|---|---|---|
| to the edge | d | the container | AT | AT / RV |
| RV | (cm) | h (cm) | (cm²) | (cm²/cm³) |
| 2 ml¹⁾ | 1.60 | 3.50 | 19.60 | 4.90 |
| 4 ml¹⁾ | 1.60 | 4.50 | 24.63 | 4.10 |
| 10 ml¹⁾ | 2.40 | 4.50 | 38.45 | 2.85 |
| 20 ml¹⁾ | 3.00 | 6.00 | 58.56 | 2.34 |
| 30 ml²⁾ | 3.60 | 6.28 | 81.21 | 2.14 |
| 50 ml³⁾ | 4.60 | 6.80 | 114.89 | 1.71 |
| 100 ml²⁾ | 5.16 | 9.45 | 174.10 | 1.46 |
| 250 ml³⁾ | 6.60 | 13.60 | 316.20 | 1.03 |

¹⁾EN ISO 8362-1; Glass vial of tubular glass Type I
²⁾EN ISO 8362-1; Glass vial from glass blanks Type I
³⁾EN ISO 58363-5 Infusion bottles from glass blanks Type I

The percentage of residual content of active ingredient in the gemcitabine preparation at the end of the running time which results from a decomposition of the active ingredient in during storage is limited to 95% of the initial content by registration guidelines of the Drug Authorities.

In this connection and in the following stability investigations, the term “stability of the solution” is to be understood to be the stability of the gemcitabine solution, that is, as the long-term consistency of the concentration of the starting compound after it is brought into solution.

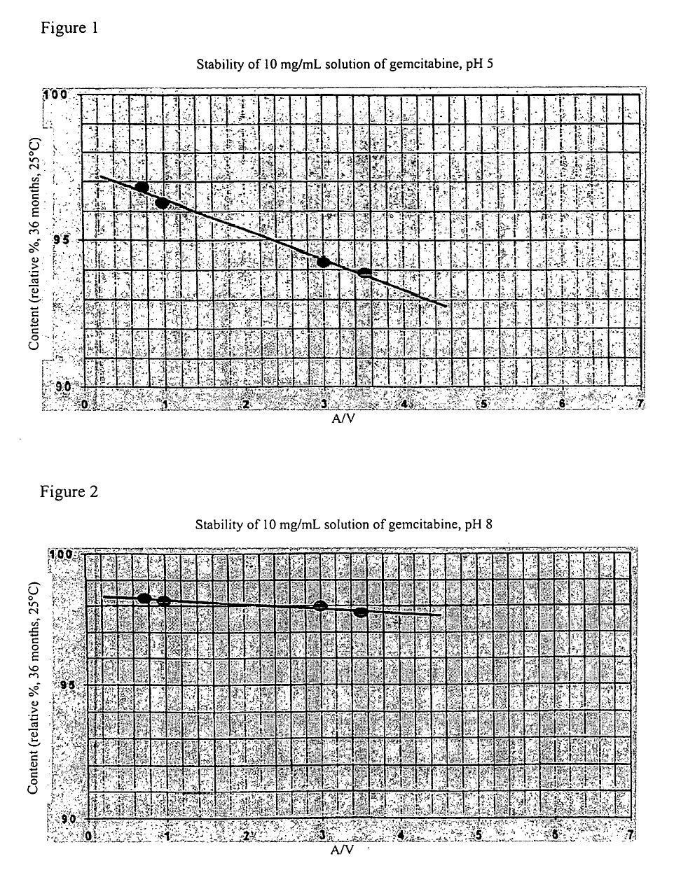

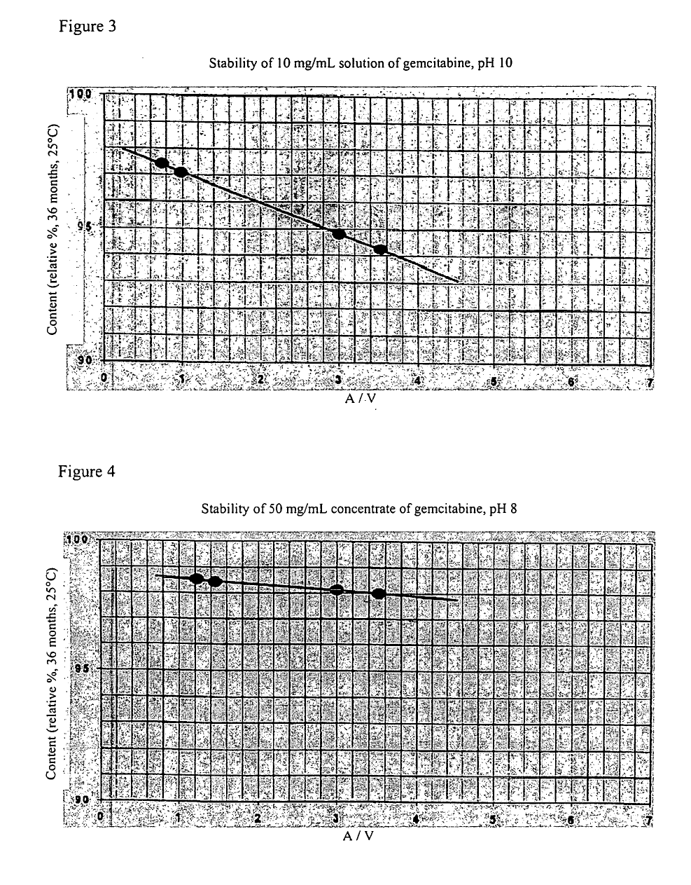

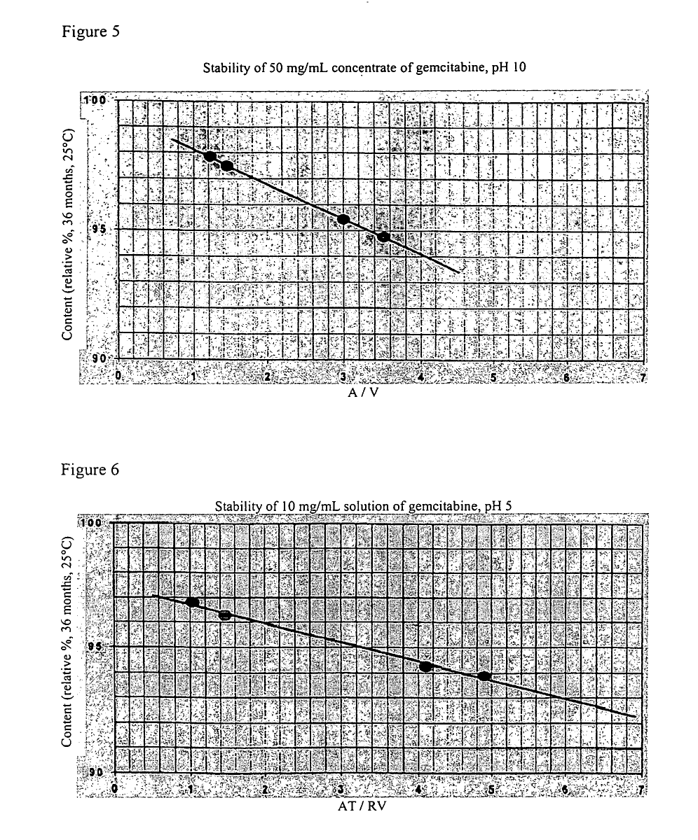

The stability investigations of ready-to-use solutions containing gemcitabine, which are described in greater detail in the following, led to the surprising result that gemcitabine, when dissolved in an aqueous solution and filled into a tightly closed container of glass, has a pharmaceutically suitable stability if the container is characterized by a ratio of surface area (A) wetted by gemcitabine solution, to volume (V) filled with gemcitabine solution, which is less than about 3.4 (cm²/cm³). As can be inferred from FIGS. 1-5, vessels with such an A/V ratio permit ready-to-use gemcitabine solutions and solution concentrates to be prepared which are stable over a long period, the pH of these solutions being able to vary over a wide range from 5 to 10.

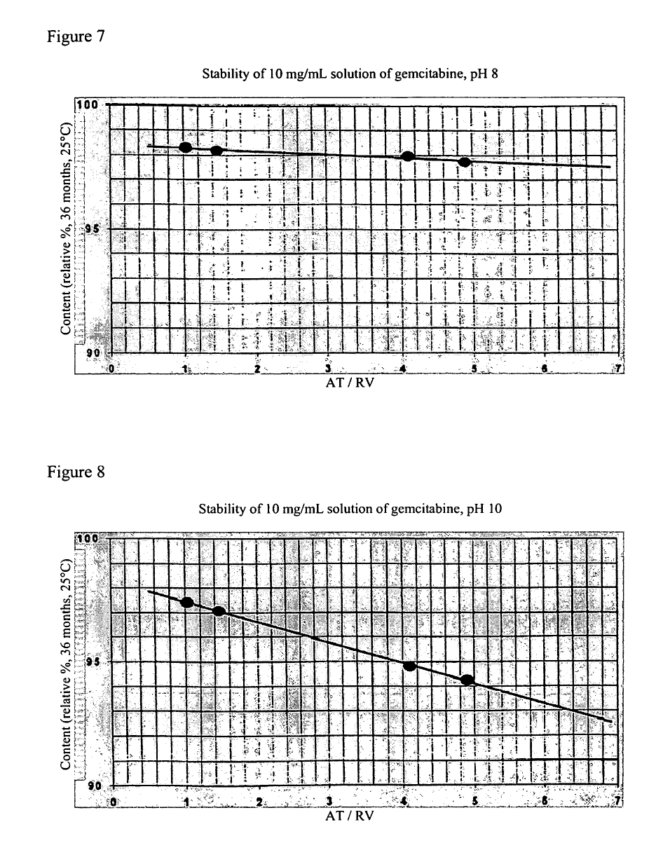

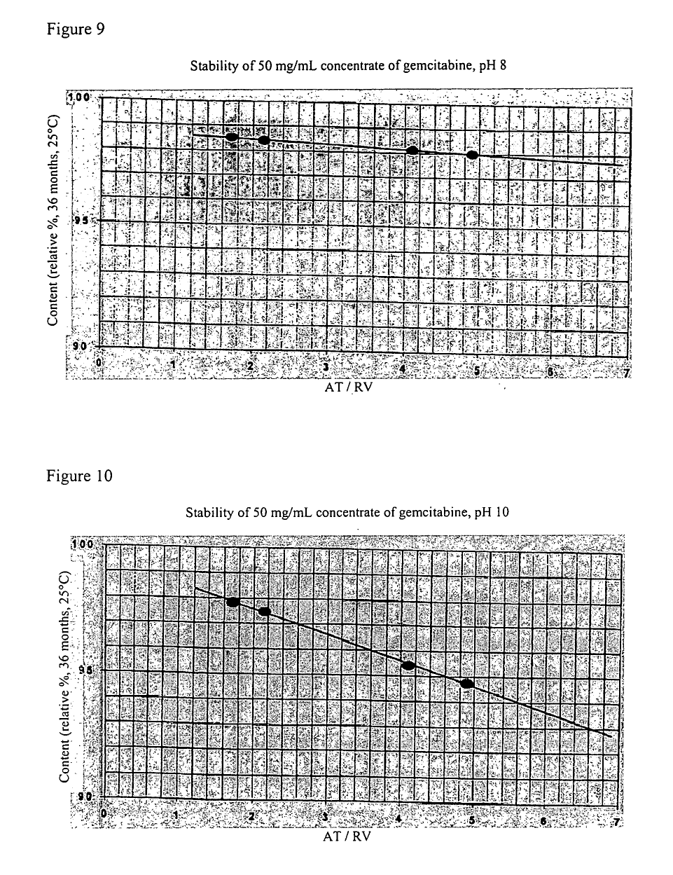

The stability investigations furthermore led to the surprising result that gemcitabine dissolved in an aqueous solution and filled into a tightly closed container of glass, has a pharmaceutically suitable stability if the container is characterized by a ratio of the surface area of the glass container to the full-to-the-edge capacity of less than about 4.6 (cm²/cm³, AT/RV). As can be inferred from FIGS. 7-10, vessels with such an AT/RV ratio permit ready-to-use gemcitabine solutions and solution concentrates to be prepared which are stable over a long period, the pH of these solutions being able to vary over a wide range from 8 to 10. As can be seen from FIG. 6, sufficient stability is attained even at a pH of 5 when the AT/VR ratio is below 3.3 (cm²/cm³).

There is no prior indication in the state of the art for this completely surprising phenomenon, which has been observed as an expression of a dependency of the stability of aqueous, dissolved gemcitabine on a geometric parameter of the container.

The inventive solutions range in concentration from 0.05 to 16.0 mg of gemcitabine per milliliter of solvent. Preferably, the concentration of the gemcitabine is 10 mg/mL. Moreover, the solutions may contain free gemcitabine base or a physiologically acceptable acid addition salt thereof. Preferably, the free gemcitabine base, and especially the acid addition salt of the gemcitabine base with an inorganic acid, particularly gemcitabine hydrochloride, is used for the preparation of the inventive solutions.

The inventive solution concentrates comprise from 16.0 mg to 110.0 mg of gemcitabine per milliliter of solvent. Preferably, the concentration of gemcitabine is 50 mg/mL or 80 mg/mL.

Suitable solvents for inventive solutions and solution concentrates are, for example, water, ethanol, glycerin, 1,2-propylene glycol, polyethylene glycol 200 to 600, benzyl alcohol, trimethylene glycol, 1,3-butylene glycol, 2,3-butylene glycol, ethyl acetate, ethyl lactate, glycofurol (tetraglycol), solketal and urea. Preferably water, ethanol, polyethylene glycol 200 to 600 or 1,2-propylene glycol, and especially water, is used as the solvent.

The pH of the inventive solutions and solution concentrates is adjusted by mixing a suitable ratio of the gemcitabine base with a physiologically acceptable acid addition salt thereof, preferably with gemcitabine hydrochloride.

However, pursuant to the invention, it is also possible to adjust the pH with at least one physiologically acceptable acidifying and/or alkalizing agent. For example, inorganic acids and bases are suitable for this purpose, such as hydrochloric acid, sulfuric acid, sulfurous acid, nitric acid, nitrous acid, phosphoric acid, phosphorous acid, carbonic acid, sodium hydroxide, potassium hydroxide, calcium hydroxide and magnesium hydroxide; alkali salts and alkaline earth salts, as well as alkali hydrogen salts and alkaline earth hydrogen salts of inorganic oxo acids of phosphorus, sulfur, carbon and nitrogen, such as sodium phosphate and its hydrate, sodium hydrogen phosphate and its hydrate, disodium hydrogen phosphate and its hydrate, disodium sulfate, sodium hydrogen sulfate, sodium sulfite, calcium sulfite, magnesium sulfite, calcium hydrogen carbonate, sodium carbonate, sodium hydrogen carbonate, sodium nitrate, sodium nitrite, calcium nitrite, magnesium nitrate and magnesium nitrite; salts of chlorine, such as sodium chloride, calcium chloride and magnesium chloride, organic bases and acids such as formic acid, acetic acid, propionic acid, lactic acid, oxalic acid, malonic acid, maleic acid, tartaric acid, citric acid, pyruvic acid, benzoic acid, methanesulfonic acid, ethanesulfonic acid, p-toluenesulfonic acid, salicylic acid, ascorbic acid, tris(hydroxymethyl-)aminomethane (2-amino-2-(hydroxymethyl)-1,3-propane diol (Trometamol; TRIS), 1-desoxy-(methylamino)-D-glucitol (N-methylglucamine; Meglumin); alkali salts and alkaline earth salts of organic bases and acids such as sodium acetate; as well as mixtures thereof.

Preferably, the pH of the inventive solutions and solution concentrates is adjusted with hydrochloric acid, phosphoric acid, sulfuric acid, sodium hydroxide, sodium phosphate and its hydrates, sodium hydrogen phosphate and its hydrates, disodium hydrogen phosphate and its hydrates, acetic acid, lactic acid, citric acid, methanesulfonic acid, methanesulfonic acid, ethanesulfonic acid, tris(hydroxymethyl-)aminomethane (Trometamol; TRIS) or 1-desoxy-(methylamino)-D-glucitol (N-methylglucamine; Meglumin), especially with sodium hydroxide, hydrochloric acid, tris(hydroxymethyl-)aminomethane (Trometamol; TRIS) or 1-desoxy-(methylamino)-D-glucitol (N-methylglucamine; Meglumin).

The pH can also be adjusted and/or stabilized by a buffer formed from physiologically acceptable acidifying and/or alkalizing agents. Especially preferred buffers for adjusting the pH of the inventive solutions are sodium acetate, tris(hydroxymethyl-) aminomethane (Trometamol; TRIS), 1-desoxy-(methylamino)-D-glucitol (N-methylglucamine; Meglumin) and disodium hydrogen phosphate or mixtures thereof.

The inventive solutions and solution concentrates optionally additionally comprise at least one tonicizing and/or at least one preservative material.

As a tonicizing agent for the inventive solutions and solution concentrates, physiologically acceptable inorganic alkali or alkaline earth salts such as sodium chloride, calcium chloride, magnesium chloride, sodium sulfate, sodium carbonate and calcium hydrogen carbonate; physiologically acceptable organic salts such as sodium lactate; physiologically acceptable carbohydrates, such as glucose, fructose, sorbitol, mannitol, galactose, inositol, maltitol, lactose, trehalose, maltose, sucrose, dextran 1, dextran 10, dextran 40, dextran 70, starch and hydroxyethyl starch; physiologically acceptable amino acids, peptides or proteins such as glycine, albumin and gelatins; as well as mixtures thereof may, for example, be used.

Preferred tonicizing agents are sodium chloride, calcium chloride, glucose, mannitol and lactose; sodium chloride, glucose and mannitol being particularly preferred.

As preservatives for the inventive solutions and solution concentrates, chlorocresol, benzyl alcohol, esters of p-hydroxybenzoic acid, such as ethyl paraben and methyl paraben, may, for example, be used.

Preferred preservatives are benzyl alcohol, ethyl paraben and methyl paraben, benzyl alcohol being particularly preferred.

Test sample batches with a gemcitabine concentration of 10 mg/mL, produced for the stability investigations, were prepared in the following manner.

To prepare the solutions, 80% of the required water for injection purposes was taken and the gemcitabine hydrochloride was added and dissolved until a clear solution was obtained. The pH was adjusted to the desired value by adding the required amount of 1N sodium hydroxide solution or 1N hydrochloric acid (maximum deviation±0.2 pH units).

The pH was determined potentiometrically. The formulation was diluted with water for injection purposes to the desired volume and the pH, if necessary, was adjusted once again with the necessary amounts of 1N sodium hydroxide solution or 1N hydrochloric acid (maximum deviation±0.2 pH units).

The test solutions were sterilized by filtration and filtered under aseptic conditions into glass containers up to their nominal volume, and the glass containers were sealed with septums, flanged airtight and subsequently protected from light for 12 months at 25° C. and, “under accelerated conditions”, at 40° C.

The pH, as a parameter of stability, was determined potentiometrically and the “content”, as another measure of stability, was determined by HPLC at the test cycles mentioned. The results of the investigations carried out are summarized in the following Table 3.

The analyses of the stability test samples gave the following results, which are summarized in Table 3.

Stability of 10 mg/mL Gemcitabine Solution

Composition

Gemcitabine (10 mg, corresponding to 11.39 mg of gemcitabine hydrochloride) dissolved in a solvent (water for injection purposes) and diluted to 1.0 mL; the pH of the solution was adjusted with 1N sodium hydroxide/1N hydrochloric acid (both for injection purposes) to values of 5.0 (±0.2) and 8.0 (±0.2) or 10.0 (±0.2).

TABLE 3
Results of the Stability Investigation and Extrapolated Stabilities

| | Strength; Packing Material | | | | | | | | | | | |
|---|---|---|---|---|---|---|---|---|---|---|---|---|
| | (20 mg) | | | (40 mg) | | | (1.0 g) | | | (2.5 g) | | |
| | 2 R¹⁾ | | | 4 R¹⁾ | | | 100 H²⁾ | | | 250 H³⁾ | | |
| | A / V | | | | | | | | | | | |
| | 3.52 | | | 3.00 | | | 0.98 | | | 0.74 | | |
| | AT / RV | | | | | | | | | | | |
| Content | 4.90 | | | 4.10 | | | 1.46 | | | 1.03 | | |
| (HPLC; | | | pH | | | pH | | | pH | | | pH |
| rel. %) | pH 5 | pH 8 | 10 | pH 5 | pH 8 | 10 | pH 5 | pH 8 | 10 | pH 5 | pH 8 | 10 |
| Temp. | 25° C. | 25° C. | 25° C. | 25° C. | 25° C. | 25° C. | 25° C. | 25° C. | 25° C. | 25° C. | 25° C. | 25° C. |
| 0 months | 100.0 | 100.0 | 100.0 | 100.0 | 100.0 | 100.0 | 100.0 | 100.0 | 100.0 | 100.0 | 100.0 | 100.0 |
| 3 months | 99.23 | 99.72 | 99.28 | 99.43 | 99.75 | 99.35 | 99.61 | 99.77 | 99.64 | 99.69 | 99.79 | 99.68 |
| 6 months | 98.97 | 99.64 | 99.10 | 99.25 | 99.68 | 99.19 | 99.49 | 99.72 | 99.55 | 99.51 | 99.73 | 99.59 |
| 9 months | 98.25 | 99.34 | 98.26 | 98.27 | 99.39 | 98.43 | 99.03 | 99.45 | 99.12 | 99.06 | 99.49 | 99.22 |
| 12 months | 97.99 | 99.27 | 98.15 | 98.19 | 99.35 | 98.33 | 98.74 | 99.42 | 99.06 | 98.99 | 99.45 | 99.17 |
| 36 months* | 93.89 | 97.72 | 94.24 | 94.25 | 97.97 | 94.80 | 96.27 | 98.19 | 97.07 | 96.80 | 98.29 | 97.41 |
| t₉₅ (mon.)** | 29 | 80 | 31 | 31 | 90 | 35 | 48 | 101 | 62 | 56 | 107 | 70 |
| Temp. | 40° C. | 40° C. | 40° C. | 40° C. | 40° C. | 40° C. | 40° C. | 40° C. | 40° C. | 40° C. | 40° C. | 40° C. |
| 0 months | 100.0 | 100.0 | 100.0 | 100.0 | 100.0 | 100.0 | 100.0 | 100.0 | 100.0 | 100.0 | 100.0 | 100.0 |
| 3 months | 97.54 | 99.19 | 97.96 | 97.78 | 99.28 | 98.16 | 98.75 | 99.36 | 98.97 | 98.89 | 99.40 | 99.08 |
| 6 months | 95.03 | 98.37 | 95.87 | 95.52 | 98.55 | 96.28 | 97.48 | 98.71 | 97.91 | 97.76 | 98.79 | 98.14 |
| 9 months | 92.38 | 97.50 | 93.68 | 93.14 | 97.77 | 94.32 | 96.14 | 98.02 | 96.80 | 96.57 | 98.14 | 97.16 |
| 12 months | 89.16 | 96.44 | 91.00 | 90.23 | 96.84 | 91.89 | 94.51 | 97.18 | 95.45 | 95.12 | 97.35 | 95.95 |
| 36 months* | 67.98 | 89.49 | 7342 | 71.15 | 90.66 | 7607 | 83.79 | 91.67 | 86.56 | 85.59 | 92.18 | 88.05 |
| t₉₅ (mon.)** | 6 | 17 | 7 | 6 | 19 | 8 | 11 | 22 | 14 | 13 | 23 | 15 |

*Extrapoluted,
**Values rounded off (month = 30 days)
¹⁾EN ISO 8362—Glass vial of tubular glass Type I
²⁾EN ISO 8362—Glass vial from glass blanks Type I
³⁾EN ISO 58363-5 Infusion bottles from glass blanks Type I

At storage temperatures of 25° C. and 40° C. for a period of 12 months, the solutions remained clear, colorless and free of visible particles and crystalline formations.

Test sample batches for the stability investigations were prepared with a gemcitabine concentration of 50 mg/mL in the following manner:

To prepare the solutions in Table 4, water for injection purposes was added in 90% of the required amount, followed by absolute ethanol as a further solvent also in 90% of the required amount. The formulation was heated, 95% of the required amount of the appropriate pH-adjusting material (such as 5N sodium hydroxide solution/5N hydrochloric acid) was added and the formulation was heated once again.

The gemcitabine hydrochloride was added in an amount corresponding to 100% of that required and stirred until a clear solution was obtained, the pH being determined potentiometrically and adjusted with the required amount of the appropriate alkalizing agent (such as 5N sodium hydroxide solution/5N hydrochloric acid) to the desired pH. The formulation was made up to volume with the required amount of water for injection purposes and with the further solvent (absolute ethanol).

The heated formulation was stirred until a clear solution was obtained. The pH was checked once again and, if necessary, adjusted to the desired value with sodium hydroxide solution or hydrochloric acid. The formulation was cooled to room temperature and filtered sterile into a sterile container. The solution concentrate obtained was filled under aseptic conditions into glass containers up to their nominal volume. The flasks, sealed with a septum, were protected against light and kept at 25° C. and, “under accelerated conditions” at 40° C. Samples for analysis were analyzed for purity (by means of HPLC) and pH (potentiometrically) at the appropriate times.

The analysis of the stability test samples gave the results summarized in the following Table 4.

The Stability of 50 mg/mL Gemcitabine Concentrate

Composition

Gemcitabine (50 mg, corresponding to 56.95 mg of gemcitabine hydrochloride) dissolved in solvent (0.500 mL anhydrous ethanol, 0.500 mL water for injection purposes) and diluted to 1.0 mL; the pH of the solution was adjusted with 1N sodium hydroxide/1N hydrochloric acid (both for injection purposes) to values of 5.0 (±0.2) and 8.0 (±0.2) or 10.0 (±0.2).

TABLE 4
Results of the Stability Investigation and Extrapolated Stabilities

| | Strength; Packing Material | | | | | | | |
|---|---|---|---|---|---|---|---|---|
| | (20 mg) | | (40 mg) | | (1.0 g) | | (2.5 g) | |
| | 2 R¹⁾ | | 4 R¹⁾ | | 100 H²⁾ | | 250 H³⁾ | |
| | A / V | | | | | | | |
| | 3.52 | | 3.00 | | 0.98 | | 0.74 | |
| Content | AT / RV | | | | | | | |
| (HPLC; rel. | 4.90 | | 4.10 | | 1.46 | | 1.03 | |
| %) | pH 8 | pH 10 | pH 8 | pH 10 | pH 8 | pH 10 | pH 8 | pH 10 |
| Temp. | 25° C. | 25° C. | 25° C. | 25° C. | 25° C. | 25° C. | 25° C. | 25° C. |
| 0 months | 100.0 | 100.0 | 100.0 | 100.0 | 100.0 | 100.0 | 100.0 | 100.0 |
| 3 months | 99.80 | 99.60 | 99.89 | 99.65 | 99.82 | 99.76 | 99.92 | 99.85 |
| 6 months | 99.69 | 99.08 | 99.64 | 99.19 | 99.76 | 99.56 | 99.71 | 99.60 |
| 9 months | 99.39 | 98.72 | 99.59 | 98.89 | 99.54 | 99.39 | 99.67 | 99.48 |
| 12 months | 99.37 | 98.25 | 99.35 | 98.47 | 99.49 | 99.14 | 99.51 | 99.28 |
| 36 months* | 97.98 | 94.75 | 98.12 | 95.42 | 98.42 | 97.48 | 98.53 | 97.83 |
| t₉₅ (mon.)** | 89 | 34 | 96 | 39 | 115 | 72 | 122 | 83 |
| Temp. | 40° C. | 40° C. | 40° C. | 40° C. | 40° C. | 40° C. | 40° C. | 40° C. |
| 0 months | 100.0 | 100.0 | 100.0 | 100.0 | 100.0 | 100.0 | 100.0 | 100.0 |
| 3 months | 99.21 | 98.09 | 99.26 | 98.23 | 99.19 | 99.15 | 99.37 | 98.84 |
| 6 months | 98.14 | 96.73 | 98.67 | 96.12 | 98.93 | 98.07 | 98.81 | 98.07 |
| 9 months | 97.52 | 94.20 | 97.84 | 94.14 | 98.06 | 97.19 | 98.14 | 97.09 |
| 12 months | 96.60 | 91.89 | 96.69 | 91.18 | 97.18 | 95.61 | 97.21 | 96.21 |
| 36 months* | 89.80 | 76.07 | 91.52 | 74.20 | 91.90 | 87.26 | 91.20 | 88.71 |
| t₉₅ (mon.)** | 18 | 8 | 21 | 7 | 22 | 14 | 22 | 16 |

*Extrapoluted,
**Values rounded off (month = 30 days)
¹⁾EN ISO 8362-1; Glass vial of tubular glass Type I
²⁾EN ISO 8362-1; Glass vial from glass blanks Type I
³⁾EN ISO 58363-5 Infusion bottles from glass blanks Type I

The solutions remained clear, colorless and free of visible particles and crystalline formations for a period of 12 month when kept at a temperature of 25° C. or 40° C.

### EXAMPLES 1-3

Aqueous solutions of the following compositions were prepared.

| | Example 1 | Example 2 | Example 3 |
|---|---|---|---|
| Strength | 500 mg | 1000 mg | 2000 mg |
| | (50 ml) | (100 mg) | (200 mg) |
| Glass containers | 50 H¹⁾ | 100 H²⁾ | 250 H³⁾ |
| Gemcitabine HCl | 10 mg/ml | 10 mg/ml | 10 mg/ml |
| pH | 5.5 (+/−0.2) | 8.0 (+/−0.2) | 9.5 (+/−0.2) |
| 1N Sodium hydroxide | up to pH 5.5 | up to pH 8.0 | up to pH 9.5 |
| | (+/−0.2) | (+/−0.2) | (+/−0.2) |
| 1N Hydrochloric acid | up to pH 5.5 | up to pH 8.0 | up to pH 9.5 |
| | (+/−0.2) | (+/−0.2) | (+/−0.2) |
| Trometamol | — | — | 0.20 mg/ml |
| Sodium acetate | 0.20 mg/ml | — | — |
| Sodium chloride | 6.5 mg/ml | 6.5 mg/ml | 6.5 mg/ml |
| Water for injection | to 1 ml | to 1 ml | to 1 ml |
| purposes | | | |

¹⁾EN ISO 8362-1; Glass vial of tubular glass Type I
²⁾EN ISO 8362-1; Glass vial from glass blanks Type I
³⁾EN ISO 58363-5 Infusion bottles from glass blanks Type I

### EXAMPLES 4-6

Aqueous solution concentrates of the following compositions were prepared.

| | Example 4 | Example 5 | Example 6 |
|---|---|---|---|
| Strength | 500 mg | 1000 mg | 2000 mg |
| | (10 ml) | (20 mg) | (40 mg) |
| Glass containers | 10 R¹⁾ | 20 R¹⁾ | 50 H³⁾ |
| Gemcitabine HCl | 50 mg/ml | 50 mg/ml | 50 mg/ml |
| pH | 7.0 (+/−0.2) | 7.5 (+/−0.2) | 9.0 (+/−0.2) |
| 5N Sodium hydroxide | up to pH 7.0 | up to pH 7.5 | up to pH 9.0 |
| | (+/−0.2) | (+/−0.2) | (+/−0.2) |
| 5N Hydrochloric acid | up to pH 7.0 | up to pH 7.5 | up to pH 9.0 |
| | (+/−0.2) | (+/−0.2) | (+/−0.2) |
| Sodium acetate | 0.40 mg/ml | — | — |
| 15% Ethanol in | to 1 ml | to 1 ml | to 1 ml |
| water for injection | | | |
| purposes | | | |

¹⁾EN ISO 8362-1; Glass vial of tubular glass Type I
²⁾EN ISO 8362-1; Glass vial from glass blanks Type I
³⁾EN ISO 58363-5 Infusion bottles from glass blanks Type I

We claim:

1. A pharmaceutical composition including a ready-to-use preparation of gemcitabine in an aqueous solution in a container of glass, characterized in that the ratio of the surface area of the container wetted by the composition to the volume of the composition contained in the container, expressed in cm²/cm³, is less than 3.4 and the ready-to-use preparation was not reconstituted from a solid immediately before it administration to a patient.

2. A pharmaceutical composition including a ready-to-use preparation of gemcitabine in an aqueous solution in a container of glass, characterized in that the ratio of the surface area of the glass container to the capacity of the glass container filled to the edge, expressed in cm²/cm³, is less than 4.6 and the ready-to-use preparation was not reconstituted from a solid immediately before administration to a patient.

3. The pharmaceutical composition of claim 2, wherein the ratio of the surface area of the glass container to the capacity of the glass container filled to the edge is less than 3.3.

4. The pharmaceutical composition of claim 1, 2 to 3, wherein the glass container is produced from a type I blank.

5. The pharmaceutical composition of claim 1, 2 or 3, wherein the container is produced from a type I glass tubing.

6. The pharmaceutical composition of claim 1, 2 or 3, wherein the gemcitabine concentration is between 0.05 mg/mL and 110 mg/mL.

7. The pharmaceutical composition of claim 6, wherein the gemcitabine concentration is 10 mg/mL, 50 mg/mL or 80 mg/mL.

8. The pharmaceutical composition of claim 1, 2 or 3, wherein the pH of the aqueous solution is within a range of about 5.0 to about 10.0.

9. The pharmaceutical composition of claim 1, 2 or 3, which additionally contains at least one tonicizing material and/or at least one preservative material.

10. A parenteral administration solution prepared by using the pharmaceutical composition of claim 1, 2 or 3.

11. A drug for the treatment of a tumor disease prepared by using the pharmaceutical composition of claim 1, 2 or 3.
